(19) United States
(12) Patent Application Publication
(10) Pub. No.: US 2023/0293397 A1
(43) Pub. Date: Sep. 21, 2023

(54) FRAGRANCE-STABLE COSMETIC PREPARATION

(71) Applicant: BEIERSDORF AG, Hamburg (DE)

(72) Inventors: Katrin RUPP, Hamburg (DE); Svea WISCHHOEFER, Hamburg (DE); Claudia MUELLER, Tangstedt (DE); Katharina HERWIG, Hamburg (DE); Andreas BLECKMANN, Ahrensburg (DE)

(21) Appl. No.: 18/001,738

(22) PCT Filed: Jun. 17, 2021
(86) PCT No.: PCT/EP2021/066403

§ 371 Date: Mar. 3, 2023

(30) Foreign Application Priority Data
Jul. 6, 2020 (DE) 102020208407.6

(51) Int. Cl. A61K 8/06; A61K 8/34; A61K 8/73; A61K 8/55; A61K 8/35; A61Q 19/08; A61Q 19/10

(52) U.S. Cl. CPC A61K 8/062; A61K 8/347; A61K 8/35; A61K 8/55; A61K 8/732; A61Q 19/08; A61Q 19/10; A61K 2800/34

(57) ABSTRACT

Cosmetic oil-in-water emulsion (O/W emulsion) containing a) hydroxypropyl starch phosphate and b) one or more perfumes selected from the group of the compounds of the tertiary alcohols, acyclic terpinolenes, and benzopyrones, wherein the preparation is free of carbomers and polyacrylates.

The present invention relates to a cosmetic oil-in-water emulsion (O/W emulsion) comprising hydroxypropyl starch phosphate (INCI: Hydroxypropyl Starch Phosphate) and one or more perfumes selected from the group of compounds of tertiary alcohols, acyclic terpinols and benzopyrones, wherein the preparation is free from carbomers and polyacrylates.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Skin care products, generally creams, ointments or lotions, mostly serve for moisturizing and refatting the skin. Active ingredients are commonly added thereto, which are intended to regenerate the skin and for example to prevent and reduce the premature aging thereof (e.g. the appearance of fine lines and wrinkles).

In addition to cleansing and caring for the skin, cosmetics also have an aesthetic function. They are intended to “improve” the external appearance of the user according to the respective cultural ideas. Cosmetics thus fulfil a psychological and social function, since they increase the (visual) attractiveness of the user. This area includes above all “decorative” cosmetics, which change the appearance of the user with the help of colorants applied to the skin. Indirectly, however, cleansing and care products also have a positive influence, since clean, healthy skin corresponds to people’s ideal of beauty.

Recently there has been an increasing trend towards “natural” cosmetics, the ingredients of which should if possible no longer come from petroleum products or be chemically synthesized. The search for alternative ingredients that meet these criteria poses particular challenges for product developers. This is because the replacement of known ingredients such as mineral oils, silicone oils and polyacrylates is practically always at the expense of disadvantages with respect to the application of the products. The preparations become sensorially unattractive, which is unpleasantly noticeable, for example, when spreading the preparation on the skin and in the lack of absorption capacity.

A particular problem arises when replacing polyacrylates, such as carbomers or acrylates/C10-30 alkyl acrylate crosspolymer with natural polymers, such as celluloses with regard to the persistence of the fragrance of the preparation. Perfumes are usually added to cosmetic preparations, which are intended to impart to the preparation a characteristic fragrance/smell. If carbomers are replaced with conventional cellulose derivatives, on the one hand more perfume is required to achieve the same fragrance intensity. On the other hand, it has been found that the scent changes relatively quickly under the influence of light and/or higher storage temperatures.

It was therefore the object of the present invention to develop a sensorially attractive, polyacrylate-free (in particular free from carbomers and acrylates/C10-30 alkyl acrylate crosspolymers) cosmetic preparation which has a storage-stable fragrance with high fragrance intensity using conventional use concentrations of perfumes.

Surprisingly, the object is achieved by a cosmetic oil-in-water emulsion (O/W emulsion) containing
a) hydroxypropyl starch phosphate (INCI: Hydroxypropyl Starch Phosphate),
b) one or more perfumes selected from the group of compounds of tertiary alcohols, acyclic terpinols and benzopyrones, wherein the preparation is free from carbomers and polyacrylates.

The object is also surprisingly achieved by a process for stabilizing the odor of cosmetic O/W emulsions containing perfumes, which are free from carbomers and polyacrylates, wherein hydroxypropyl starch phosphate (INCI: Hydroxypropyl Starch Phosphate) is added to the preparation.

Not least, the object is surprisingly achieved by using hydroxypropyl starch phosphate (INCI: Hydroxypropyl Starch Phosphate), in cosmetic O/W emulsions comprising perfumes, as a substitute for carbomers and polyacrylates.

In particular, it was not foreseeable to the person skilled in the art that the fragrance results would even exceed the values for carbomer in terms of stability.

With regard to the process and use, it is advantageous according to the invention if one or more compounds from the group of tertiary alcohols, acyclic terpinols and benzopyrones are used as perfumes.

In the context of the present disclosure, the phrases “according to the invention”, “advantageous according to the invention” etc. always refer to the emulsion according to the invention, the use according to the invention and the process according to the invention, unless otherwise described in the individual case.

Embodiments of the present invention which are preferred according to the invention are characterized in that one or more compounds from the group linalool, limonene, citronellol and coumarin are used as perfumes.

According to the invention, it is particularly preferred if the preparation does not contain any Lyral. In the scope of the present disclosure, this compound is explicitly excluded from the compounds of tertiary alcohols.

Embodiments of the present invention that are advantageous according to the invention are characterized in that the O/W emulsion contains hydroxypropyl starch phosphate (INCI: Hydroxypropyl Starch Phosphate) at a concentration of 0.2 to 5% by weight, based on the total weight of the preparation. Here, a concentration of 0.75 to 1.5% by weight, based on the total weight of the preparation, is preferred according to the invention.

It is also advantageous according to the invention if the O/W emulsion comprises perfumes at a total concentration of 0.000005 to 1% by weight, based on the total weight of the preparation.

The preferred use concentration according to the invention for linalool is from 0.0000050 to 0.0000150% by weight, based on the total weight of the preparation.

The preferred use concentration according to the invention for limonene is from 0.0005000 to 0.0020000% by weight, based on the total weight of the preparation.

The preferred use concentration according to the invention for citronellol is from 0.0001000 to 0.0010000% by weight, based on the total weight of the preparation.

The preferred use concentration according to the invention for coumarin is from 0.0005000 to 0.0015000% by weight, based on the total weight of the preparation.

Embodiments of the present invention that are advantageous according to the invention are characterized in that the preparation is free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), 2-ethylhexyl 2-cyano-3,3-diphenylacrylate, homomenthyl salicylate, parabens (particularly propyl and butyl paraben), methylisothiazolinone, chloromethylisothiazolinone and DMDM hydantoin, polyethylene glycol ethers or polyethylene glycol esters and is free from mineral oils, silicone oils and mineral waxes.

In the context of the present invention, it is advantageous if the preparation comprises either a combination of 4-hydroxyacetophenone and benzyl alcohol or a combination of ethylhexylglycerin and phenoxyethanol.

If the preparation contains a combination of 4-hydroxyacetophenone and benzyl alcohol, it is preferred according to the invention if 4-hydroxyacetophenone is used at a concentration of 0.05 to 1% by weight and benzyl alcohol is used at a concentration of 0.05 to 0.5% by weight, based in each case on the total weight of the preparation.

If the preparation contain a combination of ethylhexylglycerin and phenoxyethanol, it is preferred according to the invention if ethylhexylglycerin is used at a concentration of 0.01 to 1% by weight and phenoxyethanol is used at a concentration of 0.4 to 1% by weight, based in each case on the total weight the preparation.

It is advantageous according to the invention if the preparation contains one or more alkanediols from the group of the compounds pentane-1,2-diol, hexane-1,2-diol, octane-1,2-diol, decane-1,2-diol, 2-methylpropane1,3-diol.

It is also advantageous according to the invention if the preparation contains ethanol. The preferred use concentration according to the invention for ethanol is from 3 to 8% by weight, based on the total weight of the preparation.

Advantageous embodiments according to the invention are also characterized in that the preparation contains xanthan gum (INCI: Xanthan Gum).

The preferred use concentration according to the invention for xanthan gum is from 0.05 to 5% by weight, based on the total weight of the preparation.

It is particularly preferred according to the invention if the preparation does not contain atranol, chloroatranol and hydroxyisohexyl 3-cyclohexene carboxaldehyde (= Lyral, see above).

In the context of the present invention, it is also advantageous if the preparation comprises one or more lipophilic compounds from the group cocoglycerides, coco-caprylates/caprates, dicaprylyl ether and cetyl palmitate.

The preferred use concentration according to the invention for cocoglycerides is from 2 to 6% by weight, based on the total weight of the preparation.

The preferred use concentration according to the invention for coco-caprylates/caprates is from 2 to 6% by weight, based on the total weight of the preparation.

The preferred use concentration according to the invention for dicaprylyl ether is from 1 to 4% by weight, based on the total weight of the preparation.

The preferred use concentration according to the invention for cetyl palmitate is from 0.1 to 4% by weight, based on the total weight of the preparation.

In addition, the oil phase of the O/W emulsion according to the invention may comprise the lipophilic substances customary for such preparations.

It is advantageous according to the invention if the preparation contains one or more O/W emulsifier(s) selected from the group of the compounds glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate. These can be used at the usual use concentrations.

It is preferred in accordance with the invention to use one or more emulsifiers selected from the group of the compounds glyceryl stearate, glyceryl stearate SE and sodium cetearyl sulfate.

The preferred use concentration according to the invention for glyceryl stearate is from 0.5 to 6% by weight, based on the total weight of the preparation.

The preferred use concentration according to the invention for glyceryl stearate SE is from 0.5 to 4% by weight, based on the total weight of the preparation.

The preferred use concentration according to the invention for sodium cetearyl sulfate is from 0.05 to 1% by weight, based on the total weight of the preparation.

Embodiments of the present invention that are advantageous in accordance with the invention are characterized in that the preparation contains one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone; 8-hexadecene-1,16-dicarboxylic acid, thiamidol, glycerylglucose, (2-hydroxyethyl)urea, vitamin E and derivatives thereof, hyaluronic acid and/or salts thereof, and/or licochalcone A.

The preparation according to the invention may advantageously contain moisturizers. Moisturizers are substances or mixtures of substances that give cosmetic preparations the property, when applied or spread on the surface of the skin, of reducing the loss of moisture from the stratum corneum (also known as transepidermal water loss (TEWL)) and/or of having a beneficial effect on the hydration of the stratum corneum.

Advantageous moisturizers according to the present invention are, for example, glycerin, lactic acid and/or lactates, in particular sodium lactate, butylene glycol, propylene glycol, biosaccharide gum-1, glycine soya, ethylhexyloxyglycerin, pyrrolidone carboxylic acid and urea. Furthermore, it is particularly advantageous to use polymeric moisturizers from the group of polysaccharides which are soluble in water and/or swellable in water and/or gellable with the aid of water. Particularly advantageous, for example, are hyaluronic acid, chitosan and/or a fucose-rich polysaccharide, which is listed in Chemical Abstracts under the registration number 178463-23-5 and is obtainable, for example under the name Fucogel®1000 from SOLABIA S.A. It is also possible to use moisturizers advantageously as anti-wrinkle agents, for protection against skin changes which occur during ageing of the skin for example.

The cosmetic preparations according to the invention may also advantageously, although not mandatorily, contain fillers which further improve, for example, the sensory and cosmetic properties of the formulations and which generate or enhance, for example, a velvety or silky skin feel. Advantageous fillers in the context of the present invention are starch and starch derivatives (such as tapioca starch, distarch phosphate, aluminum or sodium starch octenyl succinate and the like), pigments, which have principally neither UV filter nor staining effects (e.g., boron nitride etc.) and/or Aerosils® (CAS No. 7631-86-9) and/or talc.

It is preferred in accordance with the invention if the preparation according to the invention comprises tapioca starch and/or distarch phosphate.

The water phase of the preparations according to the invention may advantageously comprise customary cosmetic auxiliaries, such as alcohols, in particular those having a low number of carbon atoms, preferably ethanol and/or isopropanol or polyols of low carbon number and ethers thereof, preferably propylene glycol, glycerin, electrolytes, self-tanners, etc.

### Determination of Odor Stability

The odor stability according to the invention was determined as follows:

The preparations are prepared and stored at room temperature, +6° C., 40° C. and in the light. Storage is carried out in a packaging material (lotion bottle) made of polyethylene (PE). After 2, 4, 6 and 8 months, these samples are subjected to an odor test. For this purpose, after temperature-controlling at room temperature, a walnut-sized amount of the relevant preparation is placed on cardboard cards. The different samples are compared with each other by folding the cards in the middle and smelling of the preparation by a panel of trained olfactory experts. The intensity and changes in scent are compared across a 100% scale. The sample from the refrigerator serves as reference. The samples are compared and assessed by n = 6 employees. The samples are then sorted into the appropriate storage rooms.

During this assessment, it was noticed that the formulations with hydroxypropyl starch phosphate (INCI: Hydroxypropyl Starch Phosphate) are odor-stable (storage-stable fragrance with high fragrance intensity).

### EXAMPLES

The examples below are intended to illustrate the present invention without limiting it. Unless otherwise stated, all quantitative data, fractions, and percentages are based on the weight and the total amount or on the total weight of the preparations.

Example formulations

| INCI | Comparative test 1 | Comparative test 2 | Comparative test 3 | Comparative test 4 |
|---|---|---|---|---|
| Coco-glycerides | 3 | 0 | 3 | 3 |
| Cetyl Palmitate | 0.5 | 0.5 | 0.5 | 0.5 |
| Coco-Caprylate/Caprate | 3 | 0 | 3 | 3 |
| Dicaprylyl Ether | 1.5 | 0 | 1.5 | 1.5 |
| Paraffinum Liquidum | 0 | 7.5 | 0 | 0 |
| Glyceryl Stearate SE | 2 | 2 | 2 | 2 |
| Glyceryl Stearate | 0.5 | 0.5 | 0.5 | 0.5 |
| Sodium Cetearyl Sulfate | 0.15 | 0.15 | 0.15 | 0.15 |
| Glycerin | 7 | 7 | 7 | 7 |
| Alcohol Denat. + Aqua | 5 | 5 | 5 | 5 |
| Cetearyl Alcohol | 4 | 4 | 4 | 4 |
| Carbomer | 0 | 0 | 0.1 | 0.1 |
| Xanthan Gum | 0.1 | 0.1 | 0 | 0 |
| Hydroxypropyl Starch Phosphate + Aqua | 0.75 | 0.75 | 0 | 0 |
| Preservative | qs | qs | qs | qs |
| Aqua + Sodium Hydroxide | 0 | 0 | Addition to pH 6.5-7.5 | Addition to pH 6.5-7.5 |
| Citric Acid | Addition to pH 6-7 | Addition to pH 6-7 | 0 | 0 |
| Perfume | qs | qs | qs | qs |
| Linalool | 0.000009 | 0.000009 | 0.000009 | 0.000009 |
| Limonene | 0.00084 | 0.00084 | 0.00084 | 0.00084 |

1-15. (canceled)

16. A cosmetic oil-in-water emulsion (O/W emulsion), wherein the emulsion is free from carbomers and polyacrylates and comprises:
(a) hydroxypropyl starch phosphate, and
(b) one or more perfumes selected from tertiary alcohols, acyclic terpinols and benzopyrones.

17. The emulsion of claim 16, wherein (b) comprises one or more of linalool, limonene, citronellol, and coumarin.

18. The emulsion of claim 16, wherein (b) comprises at least three of linalool, limonene, citronellol, and coumarin.

19. The emulsion of claim 16, wherein (b) comprises linalool, limonene, citronellol, and coumarin.

20. The emulsion of claim 16, wherein the emulsion comprises from 0.2% to 3% by weight of (a), based on a total weight of the emulsion.

21. The emulsion of claim 16, wherein the emulsion comprises from 0.75% to 1.5% by weight of (a), based on a total weight of the emulsion.

22. The emulsion of claim 16, wherein the emulsion comprises (b) at a total concentration of from 0.000005% to 1% by weight, based on a total weight of the emulsion.

23. The emulsion of claim 16, wherein the emulsion is free from 3-(4-methylbenzylidene)camphor, 2-hydroxy-4-methoxybenzophenone, 2-ethylhexyl 4-methoxycinnamate, 2-ethylhexyl 2-cyano-3,3-diphenylacrylate, homomenthyl salicylate, parabens, methylisothiazolinone, chloromethylisothiazolinone, DMDM hydantoin, polyethylene glycol ethers, and polyethylene glycol esters.

24. The emulsion of claim 16, wherein the emulsion is free from mineral oils, silicone oils and mineral waxes.

25. The emulsion of claim 16, wherein the emulsion comprises a combination of 4-hydroxyacetophenone and benzyl alcohol.

26. The emulsion of claim 16, wherein the emulsion comprises a combination of ethylhexylglycerin and phenoxyethanol.

27. The emulsion of claim 16, wherein the emulsion further comprises one or more of pentane-1,2-diol, hexane-1,2-diol, octane-1,2-diol, decane-1,2-diol, 2-methylpropane-1,3-diol.

28. The emulsion of claim 16, wherein the emulsion further comprises ethanol.

29. The emulsion of claim 28, wherein the emulsion further comprises from 3% to 8% by weight of ethanol, based on a total weight of the emulsion.

30. The emulsion of claim 16, wherein the emulsion further comprises xanthan gum.

31. The emulsion of claim 16, wherein the emulsion does not contain atranol, chloroatranol and hydroxyisohexyl 3-cyclohexene carboxaldehyde.

32. The emulsion of claim 16, wherein the emulsion further comprises one of more compounds selected from cocoglycerides, coco-caprylates/caprates, dicaprylyl ether and cetyl palmitate.

33. The emulsion of claim 16, wherein the emulsion further comprises one or more emulsifiers selected from glyceryl stearate, glyceryl stearate SE and sodium cetearyl sulfate.

34. A method of stabilizing the odor of a cosmetic O/W emulsion which comprises perfumes and is free from carbomers and polyacrylates, wherein the method comprises adding to the emulsion hydroxypropyl starch phosphate in an amount which is effective in stabilizing the odor of the emulsion.

35. The method of claim 34, wherein the emulsion comprises as perfumes one or more compounds selected from tertiary alcohols, acyclic terpinols and benzopyrones.
